(19) United States
(12) Patent Application Publication
(10) Pub. No.: US 2010/0168517 A1
(43) Pub. Date: Jul. 1, 2010

(54) ENDOSCOPE AND A METHOD FOR FINDING ITS LOCATION

(75) Inventors: Han Bo Shim, Seongnam-si (KR); Jung Jin Hwang, Daejeon (KR); Kwang Seop Kim, Daejeon (KR); Young Dae Seo, Daejeon (KR); Byung Hyuk Kim, Daejeon (KR); Yong Woo Lee, Daejeon (KR); Chul Cha, Daejeon (KR); Bong Ki Baek, Daejeon (KR)

Correspondence Address: Workman Nydegger;1000 Eagle Gate Tower, 60 East South Temple, Salt Lake City, UT, 84111, US

(73) Assignees: INTROMEDIC CO., LTD., Seoul (KR); I3SYSTEM, Daejeon (KR)

(21) Appl. No.: 12/442,836

(22) PCT Filed: Sep. 28, 2006
(86) PCT No.: PCT/KR2006/003893

§ 371 Date: Mar. 25, 2009

(51) Int. Cl. A61B 1/00

(52) U.S. Cl. 600/117

(57) ABSTRACT

An endoscope and method for determining a position of an endoscope body within the human body are disclosed. The endoscope (100) includes at least one first electrode (200) provided in an endoscope body and adapted to generate and transmit an electric signal, at least one second electrode adapted to receive the electric signal transmitted from the first electrode (200), a database for storing electric potential values depending on positions of the endoscope body, and a controller for determining the position of the endoscope body by comparing the electric signal with the electric potential values. The endoscope can recognize the position of the endoscope within the internal organs of the human body with the use of the electric signal induction arrangement and thus, can recognize accurate positions of illness symptoms of the internal organs.

### TECHNICAL FIELD

The present invention relates to an endoscope, and more particularly, to an apparatus and method for determining a position of an endoscope body within the human body.

### BACKGROUND ART

Endoscopes are instruments designed to be inserted into the internal organs for visually examining the interior of the organs, for which it is impossible for a medical examiner to directly check illness symptoms thereof without performing a surgical operation or autopsy. The endoscopes may be classified into a type of using a single cylinder that is designed to allow a medical examiner to visually exam the interior of the internal organs, a type of using a lens system, a type of using a camera that is designed to be directly inserted into the internal organs, and a type of using a fiberscope, and the like that is made of glass-fibers. Nowadays, the endoscopes show an outstanding development in relation with digestive organs, more particularly, in relation with the stomach and generally, stomach cameras and stomach fiberscope are referred to as endoscopes.

However, it is well known that the above described various kinds of conventional endoscopes cause pain, unpleasantness, and any other problems to patients during an endoscopic examination and therefore, many patients often want to receive a medicinal therapy, etc., instead of the endoscopic examination. Accordingly, for the sake of compensating for the above described problems and, in particular, for accomplishing the diagnosis of diseases related to the small intestine that is the longest one of digestive organs, capsule type endoscopes have been developed.

Specifically, a conventional capsule type endoscope is a micro-endoscope in the form of a capsule. In use, if a patient swallows the capsule type endoscope like a medicinal pill, the capsule type endoscope is introduced into the digestive organs, such as the stomach, the small intestine, and the like. As a result, a medicinal examiner including a doctor can visually exam the interior of the digestive organs of the patient by use of a video screen, computer monitor, or the like.

However, the above described conventional capsule type endoscope has the following problems.

If the capsule type endoscope inserted in the internal organs of the human body diagnoses an illness symptom of the organ in the course of visually examining the interior of the organ, careful investigation of the illness symptom has to be performed and successively, certain medical treatments of the problematic organ have to be accomplished via a surgical operation, etc. However, in the case of the conventional capsule type endoscope, although it can diagnose illness symptoms of the internal organs, it is impossible to accurately recognize a position of the capsule type endoscope within the interior of the organs. As a result, the conventional capsule type endoscope has no ability of informing accurate positions of the illness symptoms.

Furthermore, conventionally, a wireless communication system has been used between the capsule type endoscope and an external receiver, to perform an RF modulation for the transmission of data. In the case where data is transmitted without being modulated, the receiver has a necessity for a large-scale antenna and thus, suffers from low utility thereof. Accordingly, it is essential to modulate data to have a high frequency required to achieve a communication between the capsule type endoscope and the receiver.

More specifically, in the above described RF communication manner, when using a low frequency wave as a carrier wave, there is a necessity for an excessively large antenna, but the large-scale antenna is impossible for realization. Moreover, the RF communication manner has a necessity for a higher carrier frequency for accomplishing high-speed transmission of data, and therefore, has a difficulty in the realization of a modem module. In relation with communication module systems using a micro-robot, such as the capsule type endoscope, the above described RF communication manner may cause a heavy burden in views of the size and consumption of electric power. In addition to the above described problems, due to a limited wireless frequency resource, the RF communication manner has a high possibility of interference and results in a limit in use thereof.

### DISCLOSURE OF INVENTION

An object of the present invention devised to solve the problems lies on an endoscope having an electric signal induction type communication apparatus or using the conduction of signals in a medium instead of an RF communication manner and a method for determining a position of the endoscope.

Another object of the present invention devised to solve the problems lies on a capsule type endoscope capable of accomplishing three-dimensional recognition of a position thereof and a method for determining the position of the endoscope.

The object of the present invention can be achieved by providing an endoscope comprising: at least one first electrode provided at an endoscope body and adapted to generate and transmit an electric signal; at least one second electrode adapted to receive the electric signal transmitted from the first electrode; a database for storing electric potential values depending on positions of the endoscope body; and a controller for comparing the electric signal with the electric potential values, so as to determine a position of the endoscope body.

Preferably, the controller may determine the position of the endoscope body by calculating a distance and an angle between the endoscope body and the second electrode.

In another aspect of the present invention, provided herein is a method for determining a position of an endoscope comprising: preparing a database storing electric potential values depending on positions of an endoscope body; generating and transmitting an electric signal informing a position of the endoscope body; and determining the position of the endoscope body by receiving the electric signal and comparing the electric signal with the electric potential values.

Preferably, the electric signal may be an electric potential value of the first electrode depending on the position of the endoscope body at certain places within the human body.

Preferably, the electric potential values stored in the database are obtained by calculating electric potential values when the first electrode is located at certain places within the human body.

In accordance with the above described present invention, through the use of an electric signal induction type communication apparatus or using the conduction of signals in a medium instead of an RF communication manner, it is possible to determine a position of the endoscope within the human body while accomplishing three dimensional recognition of the position.

### BRIEF DESCRIPTION OF DRAWINGS

The accompanying drawings, which are included to provide a further understanding of the invention, illustrate embodiments of the invention and together with the description serve to explain the principle of the invention.

In the drawings:

FIG. 1 is a view illustrating an endoscope according to an embodiment of the present invention.

FIG. 2 is a block diagram of an endoscope according to another embodiment of the present invention.

FIG. 3 is a view diagrammatically illustrating the configuration and operation of transmitting electrodes provided in an endoscope body.

FIGS. 4A and 4B are views diagrammatically illustrating different information recognition concepts related to a position of an endoscope body, which are accomplished on the basis of transmitting electrodes.

FIG. 5A is a two-dimensional graph illustrating contour lines of electric potential values, which are generated by two electric dipoles provided in a capsule type endoscope.

FIG. 5B is a three-dimensional graph illustrating contour lines of electric potential values, which are generated by two electric dipoles provided in a capsule type endoscope.

FIG. 6 is a flowchart illustrating a method for determining a position of an endoscope according to an embodiment of the present invention.

FIGS. 7 to 11B are views illustrating a series of procedures for determining a position of an endoscope according to an embodiment of the present invention.

### BEST MODE FOR CARRYING OUT THE INVENTION

Reference will now be made in detail to the preferred embodiments of the present invention, examples of which are illustrated in the accompanying drawings. Wherever possible, the same reference numbers will be used throughout the drawings to refer to the same or like parts.

In brief, an endoscope according to the present invention has a feature in that at least one transmitting electrode is provided in an endoscope body and adapted to generate an electric signal and transmit the electric signal to at least one receiving electrode located at the outside of the endoscope body. Preferably, the electric signal contains information related to a position of the endoscope. More preferably, the electric signal contains an electric potential value on the basis of the configuration of the transmitting electrode. If the receiving electrode receives the electric signal, a controller compares the electric signal with theoretical electric potential values stored in a database, thereby determining the position of the endoscope within the intestine.

The above described electric signal induction type communication apparatus for determining a position of the endoscope may be applied to a stomach fiberscope, etc., in addition to a capsule type endoscope.

Preferably, instead of providing the at least one transmitting electrode, two or more transmitting electrodes may be provided to accomplish three-dimensional recognition of the position of the endoscope that will be described hereinafter.

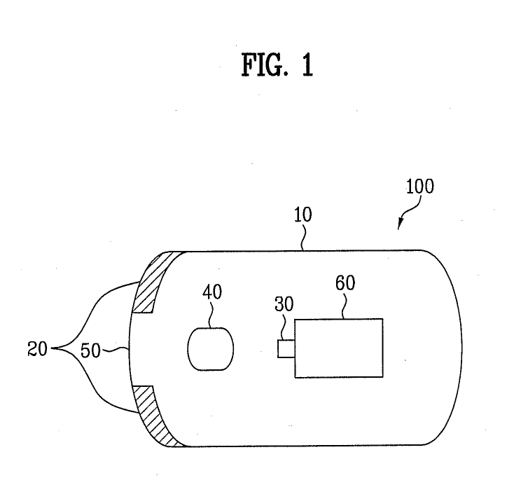

FIG. 1 is a view illustrating an endoscope according to an embodiment of the present invention. Now, the endoscope according to the embodiment of the present invention will be explained with reference to FIG. 1.

The endoscope 100 according to the present invention is preferably a wireless capsule type endoscope. More specifically, the endoscope 100 comprises a body 10, a light source 20, a camera 30, a lens 40, an aperture 50, and an image processor 60. The body 10 serves to receive a plurality of constituent elements, including the camera 30, therein, so as to integrally secure all the constituent elements. It is noted that the body 10 may have a capsule shape, or any other shape suitable to receive the internal constituent elements of the endoscope 100. Also, the body 10 may have a circular or polygonal cross section, or any other cross section. In the most preferable example, for the sake of guaranteeing easy introduction into the human body without pain, the body 10 may take the form of a capsule having a circular cross section.

The camera 30 is used to picture an image of an object to be examined, such as the digestive organs of the human body, and the like. The camera 30 may preferably use an image sensor, such as a charged-coupled device (CCD), complementary metal-oxide semiconductor (CMOS), or the like, but the present invention is not limited thereto, and any other optical appliances may be used. The lens 40 is used to project a light beam introduced thereinto to the camera 30. Here, the light beam is emitted from the object to be examined to thereby be introduced into the lens 40 through the aperture 50. Although FIG. 1 illustrates a convex lens, it will be appreciated that any other kinds of lenses are usable so long as they can regulate a focus of the image of the object to be examined, etc.

The aperture 50 is a space defined in the light source 20. Although the aperture 50 of the present embodiment has a circular shape, it will be appreciated that the aperture 50 may have other shapes including polygonal shapes. To prevent impurities, etc. from entering the body 10 of the endoscope 100, it is preferable that the aperture 50 be provided with a transparent substrate, and the like, which is mounted in an inner space of the light source 20.

The image processor 60 serves to transmit image information captured by the camera 30, or to receive and process the user's command. Specifically, the image processor 60 is able to directly transmit a pictured image of the object to the user's server, etc., or transmit the pictured image after performing a certain process, such as compression, etc. In consideration of a small size and light weight of the capsule type endoscope, preferably, the image processor 60 may have only a transmission function. In addition to the above described configuration, preferably, a drive unit for the camera 30, etc. may be provided, but detailed description thereof will be omitted because it will be clearly understood by those skilled in the art.

Although not shown in the drawings, preferably, the endoscope may further comprise a reflecting plate. Preferably, the reflecting plate is provided between the light source 20 and the body 10. With this arrangement, if a light beam is emitted from the light source 20 and advances to the body 10, the reflecting plates reflects the light beam to a forward direction of the body 10. Here, the term “forward direction” denotes a direction where most light beams emitted from the light source 20 are advanced. That is, the forward direction means a direction opposite to the advance direction of light beam from the light source 20 to the body 10. Preferably, the reflecting plate is made of epoxy containing a material having a high light-reflectivity, such as glass, etc.

Of course, it should be noted that the reflecting plate is not an essential constituent element of the present embodiment and only has a function of uniformalizing spatial distribution of light beams emitted from the light source 20 when the light beams are distributed over an excessively wide range. For example, in the case where the reflecting plate, which is made of epoxy, etc., is provided at a front side of the body 10 that is formed of a PCB, etc., the reflecting plate may act to reflect a small amount of light beams advanced from the light source 20 to the body 10, thereby regulating the intensity of illumination and the distribution of light. Preferably, the reflecting plate is configured so that an installation angle thereof is adjustable for the sake of easy regulation in the distribution of light beams.

Although not shown in FIG. 1, the body of the endoscope is preferably provided with at least one transmitting electrode. Hereinafter, the configuration and operation of the transmitting electrode will be explained.

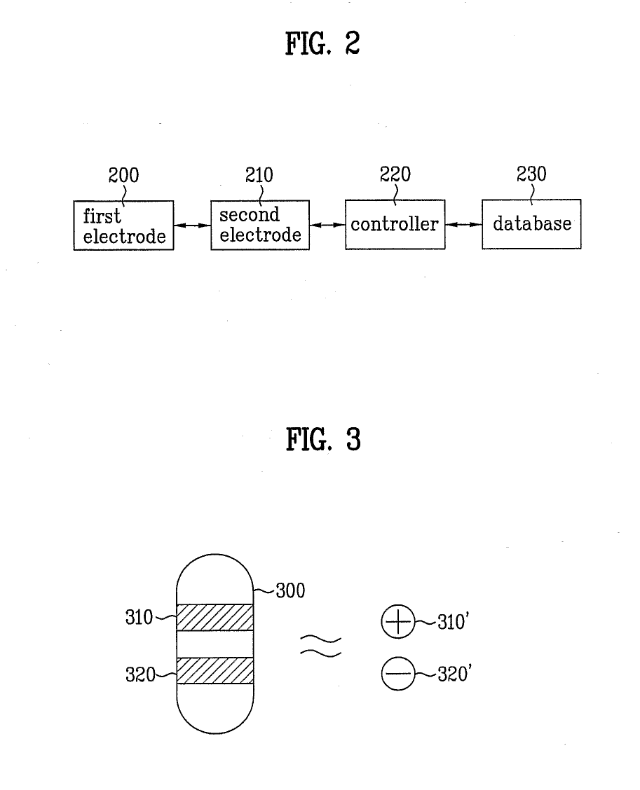

FIG. 2 is a block diagram of an endoscope according to another embodiment of the present invention. Now, the endoscope according to the present embodiment will be explained with reference to FIG. 2.

The endoscope according to the present embodiment comprises at least one first electrode 200, at least one second electrode 210, a controller 220, and a database 230. The first electrode 200 is provided at the body of the endoscope and adapted to generate and transmit an electric signal. Detailed operation of the first electrode 200 will be explained later, along with the following information related to an electric field, with reference to FIG. 3.

The second electrode 210 is adapted to receive the electric signal transmitted from the first electrode 200 and preferably, is separated from the endoscope. The database 230 serves to store theoretical electric potential values depending on different positions of the endoscope body. The controller 220 serves to compare the theoretical electric potential values stored in the database 230 with the electric signal transmitted from the transmitting electrode to the receiving electrode, so as to determine a position of the endoscope body.

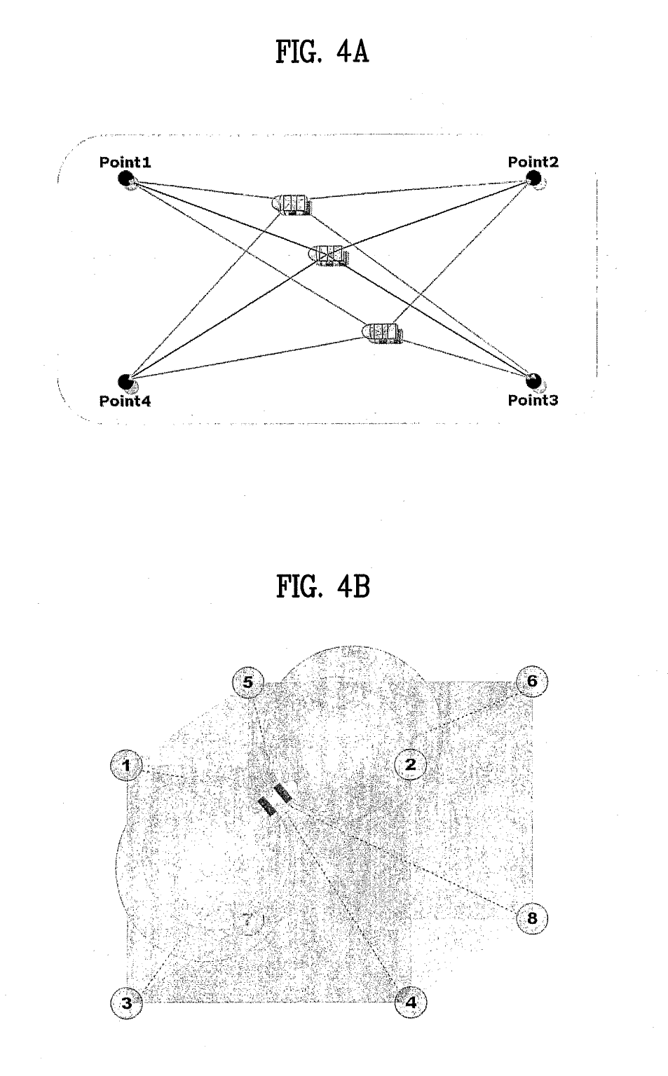

FIG. 3 is a view diagrammatically illustrating the configuration and operation of the at least one first electrode, and more particularly, two transmitting electrodes, provided at the endoscope body. FIGS. 4A and 4B are views diagrammatically illustrating different information recognition concepts related to a position of the endoscope body, which are accomplished on the basis of the transmitting electrodes. Hereinafter, different positional information recognition concepts of the endoscope according to the present invention will be explained with reference to FIGS. 3, 4A, and 4B.

Referring to FIG. 3, an endoscope body 300 is provided with two transmitting electrodes 310 and 320.

As shown in the right portion of FIG. 3, each of the transmitting electrodes 310 and 320 may be represented by two point charges. The two point charges can generate an electric potential, which may be calculated by the following equation 1.

$$V = {V}_{( + )} + {V}_{( - )} = \frac{1}{4 π {ɛ}_{0}} ( \frac{q}{{r}_{( + )}} + \frac{- q}{{r}_{( - )}} ) = \frac{q}{4 π {ɛ}_{0}} ( \frac{1}{{r}_{( + )}} - \frac{1}{{r}_{( - )}} ) = k ( \frac{1}{{r}_{( + )}} - \frac{1}{{r}_{( - )}} ) \qquad Equation 1$$

In the above Equation 1, the marks “V₍₊₎” and “V₍₋₎” represent electric potentials generated by the respective point charges, and the marks “(+)” and “(−)” represent that each of the transmitting electrodes has a positive (+) electric charge or negative (−) electric charge.

Specifically, the above described electricity information may be represented by the electric potentials represented by the Equation 1. There are provided receiving electrodes for receiving the above described positional information, more particularly, electric potential values. On the basis of the electric potential values received by the receiving electrodes, positions of the two transmitting electrodes can be calculated. As a result, a position of the endoscope body, provided with the transmitting electrodes, within the internal organs of the human body, can be calculated.

In the present invention, to reduce a time required for the calculation of the position of the endoscope body, there is provided the database for storing a variety of theoretical electric potential values depending on different distances from the receiving electrodes to the transmitting electrodes. Accordingly, by comparing electric potential values, which are transmitted from the transmitting electrodes provided at the endoscope, with the theoretical electric potential values stored in the database, it is possible to determine a position of the endoscope body.

FIG. 4A is a view illustrating a two dimensional position recognition concept and FIG. 4B is a view illustrating a three dimensional position recognition concept.

As shown in FIG. 4A, when four receiving electrodes are provided to receive electric potential values transmitted from the two transmitting electrodes provided at the endoscope, a two dimensional position of the capsule type endoscope can be recognized. Also, as shown in FIG. 4B, when eight receiving electrodes are provided to receive electric potential values transmitted from the two transmitting electrodes provided at the endoscope, a three dimensional position of the capsule type endoscope can be recognized. In this case, it is noted that the recognition of a three dimensional position is also possible by the four receiving electrodes as shown in FIG. 4, but the provision of the eight receiving electrodes as shown in FIG. 4B is more efficient to achieve a high accuracy in the recognition of a three dimensional position.

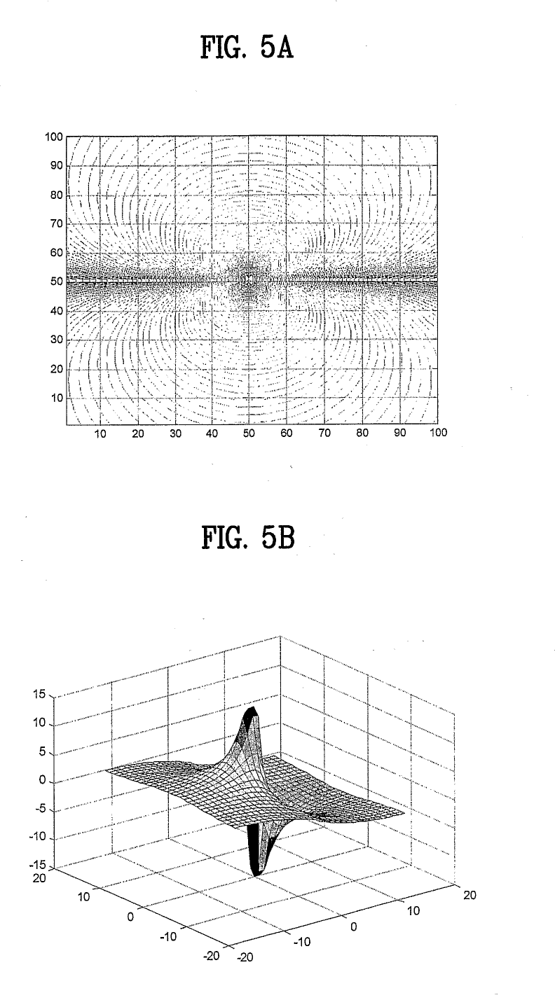

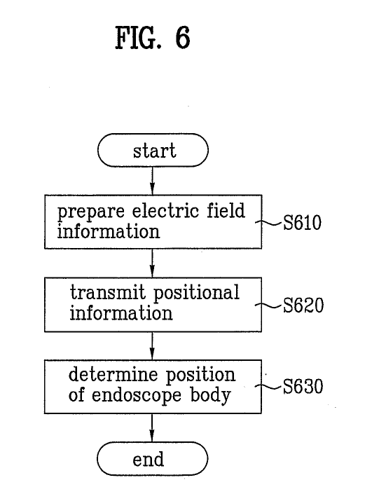

FIG. 5A illustrates voltage contour lines generated by two electric dipoles provided at the capsule type endoscope. In FIG. 5A, the voltage contour lines represent an approximately symmetrical shape about the capsule type endoscope. FIG. 5B illustrates three-dimensional voltage contour lines generated by two electric dipoles provided at the capsule type endoscope.

The controller, provided at the endoscope of the present invention, stores data related to electric potential values generated by the two transmitting electrodes (electric dipoles) provided at the endoscope body. Also, since the database stores the theoretical electric potential values depending on different positions of the endoscope body, by comparing the theoretical electric potential values stored in the database with the positional information transmitted from the transmitting electrodes provided at the endoscope that is moving in the human body, the controller can determine a position of the endoscope. Specifically, if a value coinciding with the positional information transmitted from the transmitting electrodes is found in the electric potential values stored in the database, a position of the endoscope can be found on the basis of the electric potential value by use of the graph shown in FIG. 5A or 5B, or the like.

FIG. 6 is a flowchart illustrating a method for determining a position of the endoscope according to an embodiment of the present invention. Now, the method for determining a position of the endoscope according to the embodiment of the present invention will be explained with reference to FIG. 6.

First, theoretical electric potential values depending on different positions of the endoscope body are prepared (S610). The, preparation of the theoretical electric potential values, as shown in FIGS. 5A and 5B, is performed by preparing data of theroretical electric potential values depending on relative positions between the receiving electrodes and the transmitting electrodes provided at the endoscope body. For example, by assuming that two electric dipoles are located at certain positions within the human body, theoretical electric potential values depending on different positions of the two electric dipoles can be previously calculated and stored in the database.

Then, the transmitting electrodes transmit electric signals informing a position of the endoscope body (S620). Specifically, while the endoscope body is moving in the internal organ of the human body to exam illness symptoms of the internal organ, the at least two transmitting electrodes provided at the endoscope body transmit the electric signals containing electric potential values to the receiving electrodes.

If the receiving electrodes receive the electric signals, the controller compares the electric signals with the above previously calculated and stored electric potential values, thereby determining a position of the endoscope body (S630). That is, by comparing the electric potential values prepared in the above described step S610 with the electric signals transmitted in the step S620, it is possible to find a point where the electric potential values coincide with the electric signals, and this point denotes a position of the endoscope body.

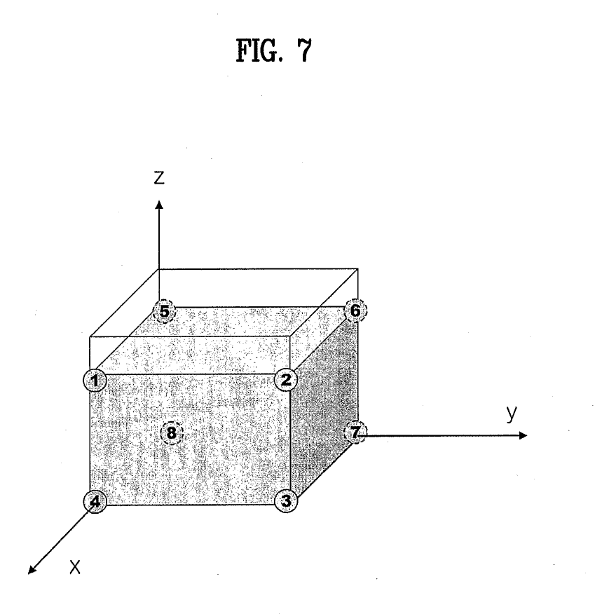

FIGS. 7 to 11B are views illustrating a series of procedures for determining a position of the endoscope according to another embodiment of the present invention. Now, the series of procedures for determining a position of the endoscope according to the present embodiment will be explained with reference to FIGS. 7 to 11B.

The determination of a position of the endoscope according to the present embodiment is performed under the assumption that receiving electrodes are provided at eight corners of a hexahedron and the endoscope provided with the two transmitting electrodes is moving in the hexahedron, as shown in FIG. 7.

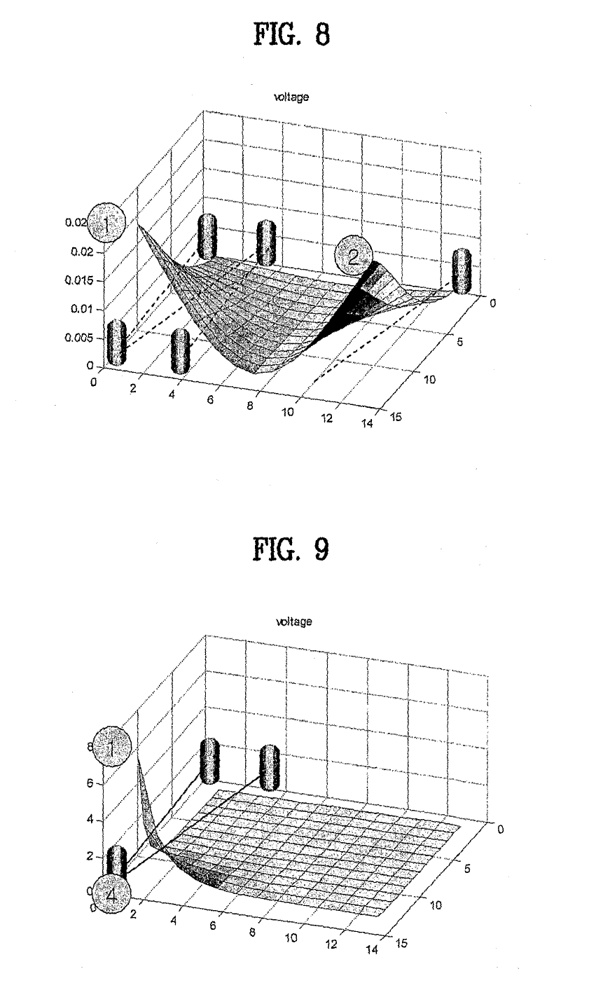

FIG. 8 illustrates the shape of signals read by a pair of receiving electrodes 1 and 2 when the endoscope body is moving along paths shown by dotted lines. FIG. 9 illustrates the shape of signals read by a pair of receiving electrodes 1 and 4. In this case, it is assumed that movement paths of the endoscope body in FIGS. 8 and 9 are the same as each other. Also, although no movement paths represented by dotted lines are shown in FIG. 8, it is noted that FIG. 8 illustrates a curve representing the variation of electric potential values in the case where the endoscope is continuously moved vertically and obliquely.

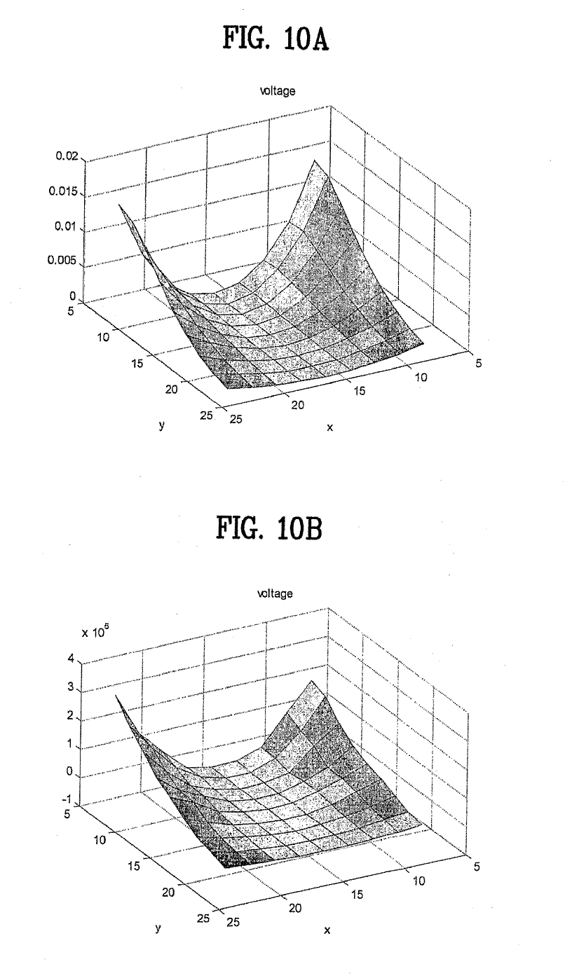

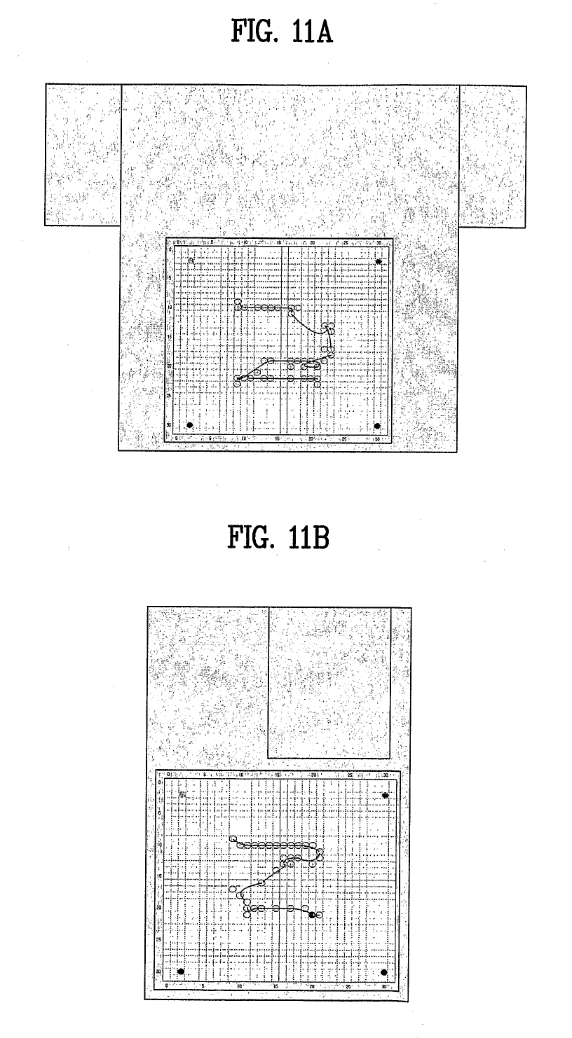

FIG. 10A illustrates a curve representing the variation of theoretical electric potential values when using eight receiving electrodes, and FIG. 10B illustrates a curve representing the variation of electric potential values received by the eight receiving electrodes while the endoscope is moving. As shown, there is no substantial difference between theoretically calculated values and experimentally obtained values. By comparing the theoretical map (FIG. 10A) with the experimental map (FIG. 10B) and taking a value representing the strongest relationship between both the maps, the value indicates a position of the capsule type endoscope. FIGS. 11A and 11B are graphs illustrating results of tracing the position of the above described endoscope within the small intestine of the human body, FIG. 11A being observed from a front side of the human body, and FIG. 11B being observed from a lateral side of the human body.

The above described endoscope and method for determining a position of the endoscope according to the present invention are applicable to a fiberscope as well as a capsule type endoscope. Also, although it is preferable that two transmitting electrodes be provided to form electric dipoles, the number of the transmitting electrodes is variable. It is noted that at least two receiving electrodes have to be provided to accomplish the recognition of a three dimensional position, and the above described endoscope may be also used in the examination of the animal's organs.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

### INDUSTRIAL APPLICABILITY

The present invention provides an endoscope and a method for determining a position of the endoscope, which are applicable to various kinds of endoscopes including a stomach fiberscope as well as a capsule type endoscope. According to the present invention, a position of the endoscope within the internal organs of the human body can be recognized by use of an electric signal induction device. By recognizing an accurate position of the endoscope, the endoscope according to the present invention can guarantee accurate recognition of positions of illness symptoms.

1. An endoscope comprising:
at least one first electrode provided at an endoscope body and adapted to generate and transmit an electric signal;
at least one second electrode adapted to receive the electric signal transmitted from the first electrode;
a database for storing electric potential values depending on positions of the endoscope body; and
a controller for comparing the electric signal with the electric potential values, so as to determine a position of the endoscope body.

2. The endoscope according to claim 1, wherein the position of the endoscope body is able to be determined based on the intensity of the electric signal.

3. The endoscope according to claim 1, wherein the endoscope is of a capsule type.

4. The endoscope according to claim 1, wherein the second electrode is separated from the endoscope body.

5. The endoscope according to claim 1, wherein the at least one first electrode comprises at least two first electrodes.

6. The endoscope according to claim 1, wherein the controller determines the position of the endoscope body by calculating a distance and an angle between the endoscope body and the second electrode.

7. The endoscope according to claim 1, wherein the electric signal is an electric potential value of the first electrode depending on the position of the endoscope body within the human body.

8. The endoscope according to claim 1, wherein the electric potential values are obtained by calculating electric potential values when the first electrode is located at certain places within the human body.

9. A method for determining a position of an endoscope comprising:
preparing a database storing electric potential values depending on positions of an endoscope body;
generating and transmitting an electric signal informing a position of the endoscope body; and
determining the position of the endoscope body by receiving the electric signal and comparing the electric signal with the electric potential values.

10. The method according to claim 9, wherein the position of the endoscope body is able to be determined based on the intensity of the electric signal.

11. The method according to claim 9, wherein the preparation of the electric potential values comprises:
calculating electric potential values of electric charges generated at certain places within the human body.

12. The method according to claim 9, wherein the generation and transmission of the electric signal informing a position of the endoscope body comprises:
providing the endoscope body with at least one first electrode; and
calculating and transmitting an electric potential value of the first electrode depending on the position of the endoscope body within the human body.

13. The method according to claim 9,
wherein the at least one first electrode comprises at least two first electrodes, and
wherein the determination of the position of the endoscope body is performed by calculating a distance and an angle between the endoscope body and at least one second electrode.
